(19) United States
(12) Patent Application Publication
(10) Pub. No.: US 2021/0236010 A1
(43) Pub. Date: Aug. 5, 2021

(54) MODEL SETTING DEVICE, BLOOD-PRESSURE MEASURING DEVICE, AND MODEL SETTING METHOD

(71) Applicant: SHARP KABUSHIKI KAISHA, Sakai City, Osaka (JP)

(72) Inventors: RIEKO OGAWA, Sakai City, Osaka (JP); YOSHIHISA ADACHI, Sakai City, Osaka (JP); YUKI EDO, Sakai City, Osaka (JP); RYOTA TOMIZAWA, Sakai City, Osaka (JP)

(21) Appl. No.: 17/053,566

(22) PCT Filed: May 10, 2019
(86) PCT No.: PCT/JP2019/018756

§ 371 Date: Nov. 6, 2020

(30) Foreign Application Priority Data
May 10, 2018 (JP) 2018-091651

(51) Int. Cl. A61B 5/021; A61B 5/00

(52) U.S. Cl. CPC A61B 5/02108; A61B 5/0059; A61B 5/7221

(57) ABSTRACT

An object of the present disclosure is to set a blood-pressure measurement model that suits each living body. A model setting device comprises: a blood-pressure obtaining unit; a pulse-wave obtaining unit that obtains pulse waves in a plurality of areas; a pulse-wave parameter determining unit that determines a plurality of pulse-wave propagation times; a blood-pressure estimation model creating unit that creates a plurality of blood-pressure estimation models; a blood-pressure estimation model evaluating unit that evaluates the blood-pressure estimation models; and a model selecting unit that selects a measurement model from among the plurality of blood-pressure estimation models, based on the evaluation performed by the blood-pressure estimation model evaluating unit.

### TECHNICAL FIELD

The present disclosure relates to a model setting device that sets blood-pressure prediction models.

### BACKGROUND ART

In recent years, technologies utilizing a pulse-wave propagation time have been available as technologies for measuring the blood pressure of a human body. For example, PTL 1 discloses a technology as described below. That is, representative colors in individual areas of two or three adjacent areas in image data are respectively determined, and fundamentals in the respective areas are extracted based on the representative colors. A difference signal between the fundamentals is determined in the adjacent areas of the plurality of areas, and pulse wave information, such as a pulse-wave propagation time, in which external noise is suppressed is obtained.

### CITATION LIST

### Patent Literature

PTL 1: International Publication No. 2016/163019 (published on Oct. 13, 2016)

### SUMMARY OF INVENTION

### Technical Problem

The vascular network, the contour, and the size of the face, and so on of a living body differ from one individual to another. Thus, an area from which pulse wave information is easily obtained differs from one individual to another. Accordingly, when pulse wave information is obtained from the same portion on all living bodies, as in the technology in PTL 1, there is a case in which the pulse wave information cannot be obtained with high accuracy, and there is a problem in that the blood pressure cannot be measured with high accuracy.

One aspect of the present disclosure has an object of realizing a model setting device and a model setting method that can set a measurement model for measuring a blood pressure, the blood-pressure measurement model being suitable for each living body.

### Solution to Problem

In order to overcome the above-described problem, a model setting device according to one aspect of the present disclosure is a model setting device that sets a measurement model for measuring a blood pressure of a living body based on pulse waves of the living body. The model setting device comprises: a blood-pressure obtaining unit that obtains a blood pressure of the living body; a pulse-wave obtaining unit that obtains the pulse waves in an area on a body surface of the living body; a pulse-wave parameter determining unit that determines a plurality of pulse wave parameters by using the pulse waves obtained by the pulse-wave obtaining unit; a blood-pressure estimation model creating unit that creates a plurality of blood-pressure estimation models for estimating a blood pressure of the living body by using the plurality of pulse wave parameters determined by the pulse-wave parameter determining unit and the living body's blood pressure obtained by the blood-pressure obtaining unit; a blood-pressure estimation model evaluating unit that evaluates the plurality of blood-pressure estimation models created by the blood-pressure estimation model creating unit; and a model selecting unit that selects at least one measurement model from among the plurality of blood-pressure estimation models, based on the evaluation performed by the blood-pressure estimation model evaluating unit.

In order to overcome the above-described problem, a model setting method according to one aspect of the present disclosure is a model setting method that sets a measurement model for measuring a blood pressure of a living body based on pulse waves of the living body. The model setting method includes: a blood-pressure obtaining process of obtaining a blood pressure of the living body; a pulse-wave obtaining process of obtaining the pulse waves in an area on a body surface of the living body; a pulse-wave parameter determining process of determining a plurality of pulse wave parameters by using the pulse waves obtained in the pulse-wave obtaining process; a blood-pressure estimation model creating process of creating a plurality of blood-pressure estimation models for estimating a blood pressure of the living body by using the plurality of pulse wave parameters determined in the pulse-wave parameter determining process and the living body's blood pressure obtained in the blood-pressure obtaining process; a blood-pressure estimation model evaluating process of evaluating the plurality of blood-pressure estimation models created in the blood-pressure estimation model creating process; and a model selecting process of selecting at least one measurement model from among the plurality of blood-pressure estimation models, based on the evaluation performed in the blood-pressure estimation model evaluating process.

### Advantageous Effects of Invention

According to one aspect of the present disclosure, it is possible to set a measurement model for measuring a blood pressure, the blood-pressure measurement model being suitable for each living body.

### BRIEF DESCRIPTION OF DRAWINGS

FIG. 1 is a block diagram showing the configuration of a blood-pressure measuring device according to a first embodiment of the present disclosure.

FIG. 2 is a diagram showing a face image of a subject body divided by a face-image dividing unit comprised by the blood-pressure measuring device.

FIG. 3 is to describe a pulse-wave propagation time determining method performed by a pulse-wave parameter determining unit comprised by the blood-pressure measuring device, (a) in FIG. 3 being a diagram showing a blood vessel of a living body, and (b) in FIG. 3 being a graph showing propagation of pulse waves.

FIG. 4 is a graph for describing a measurement-model selecting method performed by a model selecting unit comprised by the blood-pressure measuring device.

FIG. 5 is a view showing one example of a blood-pressure measurement result outputting unit comprised by the blood-pressure measuring device.

FIG. 6 is a flowchart showing one example of the flow of processing in the blood-pressure measuring device.

FIG. 7 (a) in FIG. 7 is a graph showing one example of a pulse wave waveform, and (b) in FIG. 7 is a graph showing one example of an acceleration pulse wave waveform.

FIG. 8 is a graph for describing waveform features.

FIG. 9 is a block diagram showing the configuration of a blood-pressure measuring device according to a second embodiment of the present disclosure.

FIG. 10 is a graph showing a distribution of standard deviations of errors in blood-pressure estimation models, the standard deviations being determined by a model-evaluation-index determining unit, comprised by the blood-pressure measuring device, by using data for testing.

FIG. 11 is a table showing rankings of standard deviations of errors determined by the evaluation index determining unit.

FIG. 12 is a graph showing one example of power spectra of pulse wave signals.

FIG. 13 is a table for describing a measurement-model determining method performed by a measurement-model determining unit comprised by the blood-pressure measuring device.

### DESCRIPTION OF EMBODIMENTS

### First Embodiment

One embodiment of the present disclosure will be described below in detail.

A blood-pressure measuring device 1A in the present embodiment is a contactless blood-pressure measuring device that measures (estimates) the blood pressure of a subject body, which is a living body, without contacting the subject body. The blood-pressure measuring device 1A measures the blood pressure of a subject body by using a measurement model set by a model setting device 100 described below.

(Configuration of Blood-Pressure Measuring Device 1A)

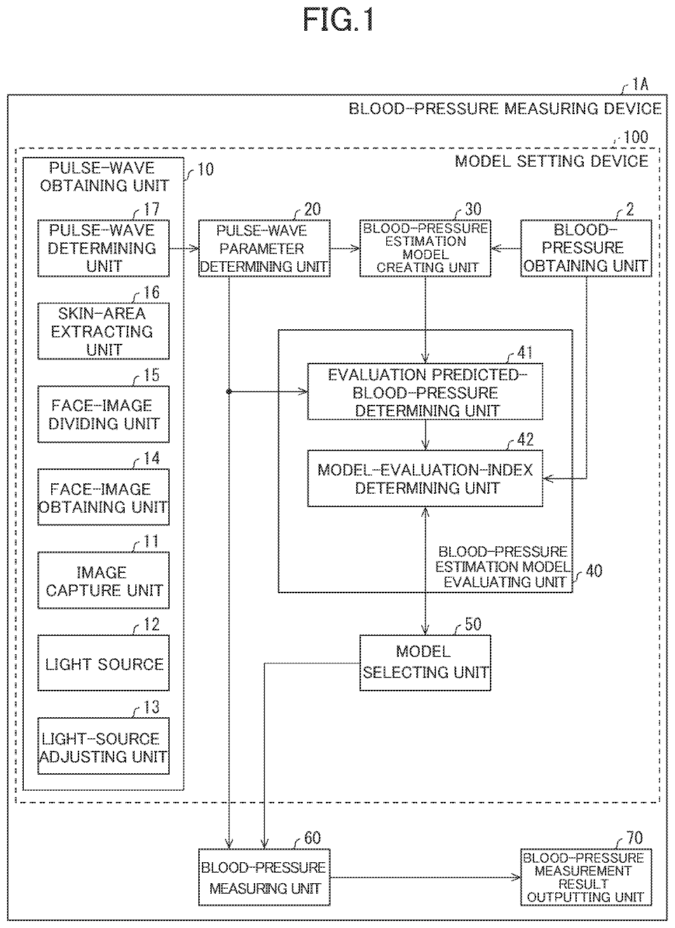

FIG. 1 is a block diagram showing the configuration of the blood-pressure measuring device 1A. As shown in FIG. 1, the blood-pressure measuring device 1A comprises a blood-pressure obtaining unit 2, a pulse-wave obtaining unit 10, a pulse-wave parameter determining unit 20 (a pulse-wave propagation time determining unit), a blood-pressure estimation model creating unit 30, a blood-pressure estimation model evaluating unit 40, a model selecting unit 50, a blood-pressure measuring unit 60, and a blood-pressure measurement result outputting unit 70.

The blood-pressure obtaining unit 2 is a contact-type sphygmomanometer that measures the blood pressure of a subject body and is, for example, a cuff sphygmomanometer. The blood pressure obtained by the blood-pressure obtaining unit 2 is used when the model selecting unit 50, which is described below, sets a measurement model. The blood-pressure obtaining unit 2 outputs the measured blood pressure of the subject body to the blood-pressure estimation model creating unit 30 and a model-evaluation-index determining unit 42, which are described below.

The pulse-wave obtaining unit 10 obtains a pulse wave at the body surface of the subject body. The pulse-wave obtaining unit 10 comprises an image capture unit 11, a light source 12, a light-source adjusting unit 13, a face-image obtaining unit 14, a face-image dividing unit 15, a skin-area extracting unit 16, and a pulse-wave determining unit 17.

The image capture unit 11 is a camera including an image sensor (e.g., a CMOS (Complementary Metal-Oxide Semiconductor), a CCD (Charge-Coupled Device), or the like) and a lens. The image capture unit 11 comprises a color filter (not shown) in an RGB Bayer arrangement that is commonly used or a color filter (not shown) for RGBCy, RGBIR, or the like which is suitable for observing an increase/decrease in the amount of blood. The image capture unit 11 captures an image of the subject body a plurality of times at predetermined time intervals (e.g., at a frame rate of 300 fps) and outputs the captured images to the face-image obtaining unit 14.

When the image capture unit 11 captures an image of the subject body, the light source 12 illuminates the subject body with light.

In order to accurately determine pulse-wave propagation times between areas used for the measurement model selected by the model selecting unit 50, which is described below, the light-source adjusting unit 13 adjusts the light source 12 so that pulse waves having certain signal qualities (e.g., pulses whose SNRs described below are high) can be detected in the corresponding areas. Specifically, the light-source adjusting unit 13 adjusts at least one of the amount of light of the light source 12, a light source spectrum, and an illumination angle relative to the skin of the subject body.

The face-image obtaining unit 14 extracts a face area of the subject body from the subject-body image, captured by the image capture unit 11, to obtain the extracted face area as a face image. By performing face tracking, the face-image obtaining unit 14 may extract, for each certain frame, a face image of the subject body, for example, from a moving image including the face of the subject body.

When the subject body puts his or her face into a set frame, and an image is acquired in a state in which the face and the camera are fixed, the face-image obtaining unit 14 can extract an image from the face of the subject body, without performing processing, such as face tracking.

The face-image dividing unit 15 divides the face image extracted by the face-image obtaining unit 14 into a plurality of areas.

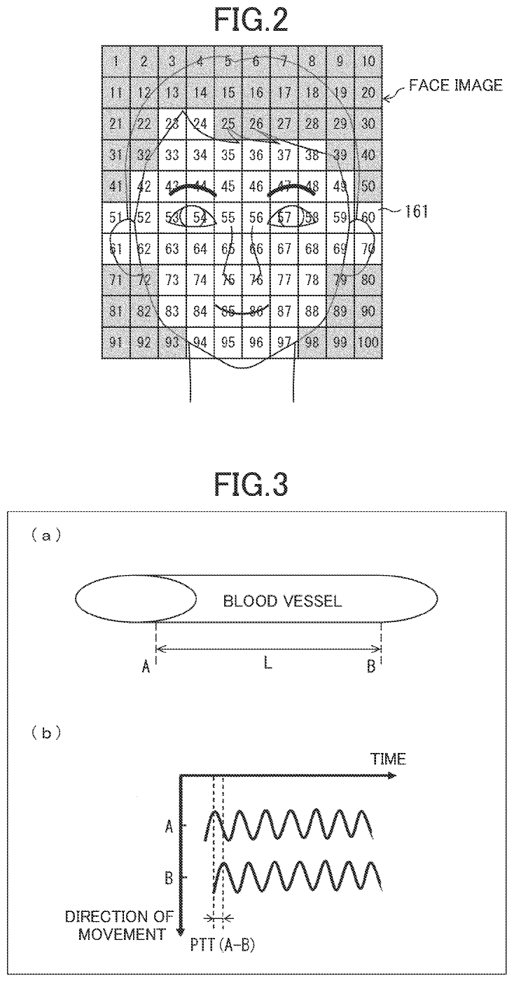

FIG. 2 is a diagram showing a subject-body's face image divided by the face-image dividing unit 15. As shown in FIG. 2, the face-image dividing unit 15 divides the face image of a subject body into 100 areas, 10 vertically by 10 horizontally. The division performed by the face-image dividing unit 15 is not limited to the above-described division method. The face-image dividing unit 15 divides the face image extracted by the face-image obtaining unit 14 into at least three areas.

The skin-area extracting unit 16 extracts, as skin areas 161, areas in which the skin is not completely covered by the hair or the like (in other words, areas in which even a part of the skin can be seen), the areas being included in the areas divided by the face-image dividing unit 15. In the example shown in FIG. 2, the areas that are not shaded are the skin areas 161, and the skin-area extracting unit 16 extracts a total of 52 portions as the skin areas 161.

The pulse-wave determining unit 17 determines pulse waves in the respective skin areas 161 extracted by the skin-area extracting unit 16. A pulse-wave determining method for the pulse-wave determining unit 17 is not particularly limiting. For example, the pulse-wave determining unit 17 determines pulse waves in the respective skin areas 161 in the manner described below.

That is, first, the pulse-wave determining unit 17 obtains signals of changes over time in average values of luminance values (pixel values) of individual colors (R, G, and B when the image capture unit 11 comprises a color filter in an RGB Bayer arrangement) in one skin area 161. Next, the pulse-wave determining unit 17 performs independent component analysis on the obtained signals to extract independent components, the number of which equals to the number of colors. Next, the pulse-wave determining unit 17 eliminates high-frequency components and low-frequency components from the extracted independent components, for example, by using a 0.75 to 4.0 Hz digital band-pass filter. Next, the pulse-wave determining unit 17 performs a fast Fourier transform on signals, obtained by eliminating the high-frequency components and the low-frequency components, to determine power spectra of frequencies of each independent component. Next, the pulse-wave determining unit 17 determines a peak (PR: Pulse Rate) of the power spectra at 0.75 to 4.0 Hz and compares the peak with a peak value of each independent component to determine the independent component having the largest peak value as a pulse wave (a pulse wave signal). The pulse-wave determining unit 17 determines pulse wave signals for the respective skin areas 161 extracted by the skin-area extracting unit 16 and outputs the determined pulse wave signals to the pulse-wave parameter determining unit 20.

By using the pulse waves (the pulse wave signals) of the skin areas 161 which are obtained by the pulse-wave obtaining unit 10, the pulse-wave parameter determining unit 20 determines pulse-wave propagation times PTT (Pulse Transit Times) between the skin areas 161 as pulse wave parameters.

FIG. 3 is to describe a pulse-wave propagation time determining method performed by the pulse-wave parameter determining unit 20, (a) in FIG. 3 being a diagram showing a blood vessel of a living body, and (b) in FIG. 3 being a graph showing propagation of pulse waves. In this case, first, a description will be given of a method for the pulse-wave parameter determining unit 20 to determine a pulse-wave propagation time PTT (A-B) between area A and area B shown in (a) in FIG. 3. First, the distance between area A and area B is represented by a distance L. The graph shown in (b) in FIG. 3 illustrates a pulse wave determined in area A and a pulse wave determined in area B. The pulse-wave parameter determining unit 20 shifts the pulse wave, determined in area A, in the time direction to determine, as the pulse-wave propagation time PTT between area A and area B, a time difference (a shift width) with which a cross-correlation coefficient between the waveform of the pulse wave determined in area A and the waveform of the pulse wave determined in area B is the largest.

The pulse-wave parameter determining unit 20 determines pulse-wave propagation times PTT for respective combinations of two areas (1326 (=52C2) combinations in total) selected from 52 areas extracted by the skin-area extracting unit 16 as the skin areas 161. For example, the pulse-wave parameter determining unit 20 determines a pulse-wave propagation time PTT (23-24) between area 23 and area 24 shown in FIG. 2.

The pulse-wave parameter determining unit 20 outputs the determined 1326 pulse-wave propagation times PTT to the blood-pressure estimation model creating unit 30, an evaluation predicted-blood-pressure determining unit 41, and the blood-pressure measuring unit 60, which are described below.

The pulse-wave parameter determining unit 20 may determine the pulse-wave propagation times PTT in more detail by performing interpolation, such as spline interpolation. Also, the pulse-wave parameter determining unit 20 may determine the pulse-wave propagation times PTT by detecting feature points, such as the maximum values of pulse waves or rise points of pulse waves, and determining a time difference between the feature points.

The blood-pressure estimation model creating unit 30 creates blood-pressure estimation models for estimating the blood pressure of the subject body, by using the pulse-wave propagation times PTT determined by the pulse-wave parameter determining unit 20 and the subject-body's blood pressure obtained by the blood-pressure obtaining unit 2, the pulse-wave propagation times PTT and the subject-body's blood pressure being data for training.

When the Young's modulus of the blood vessel is represented by E, the blood vessel wall pressure is represented by a, the blood vessel diameter is represented by R, and the blood density is represented by ρ, a speed v at which the pulse wave propagates in the blood vessel is expressed by mathematical equation 1 (the Moens-Korteweg equation) below.

$$v = \sqrt{\frac{Ea}{2 {R}_{ρ}}} \qquad [ Equation 1 ]$$

Also, the Young's modulus E of the blood vessel changes exponentially with respect to the blood pressure P. When the Young's modulus of the blood vessel for P=0 is represented by E0, the Young's modulus ER of the blood vessel is expressed by mathematical equation 2 below. In this case, γ is a constant that depends on the blood vessel.

$$P = \frac{1}{γ} ( ln \frac{1}{{T}^{2}} + ln \frac{2 R ρ {L}^{2}}{{E}_{0} a} ) \qquad [ Equation 4 ]$$

Also, when the pulse-wave propagation time is represented by T, and the blood vessel path length is L, the length L of the blood vessel path is expressed by mathematical equation 3 below.

L=vT [Equation 3]

Mathematical equation 4 below is derived from mathematical equations 1 to 3 noted above.

$$E = {E}_{0} {e}^{{γ}^{P}} \qquad [ Equation 2 ]$$

As shown in mathematical equation 4, when the length L of the blood vessel path is constant, it can be understood that the pulse-wave propagation time T has a correlation with the blood pressure P. Accordingly, the blood-pressure estimation model creating unit 30 creates blood-pressure estimation models for the blood pressure P by using the pulse-wave propagation times determined by the pulse-wave parameter determining unit 20.

Specifically, the blood-pressure estimation model creating unit 30 first creates a blood-pressure estimation model M1 with complexity 1. The “complexity” in the present disclosure means the number of explanatory variables in the blood-pressure estimation model and the number of pulse-wave propagation times used for the blood-pressure estimation model. That is, the blood-pressure estimation model M1 with complexity 1 is a blood-pressure estimation model using one pulse-wave propagation time as an explanatory variable. The blood-pressure estimation model creating unit 30 performs regression analysis using a least-squares method on one pulse-wave propagation time PTT1 determined by the pulse-wave parameter determining unit 20 and the subject body's blood pressure obtained by the blood-pressure obtaining unit 2, to thereby create the blood-pressure estimation model M1, which is a linear model noted in equation (1) below:

BP1=α1PTT1+α2 (1)

where BP1 is a predicted blood pressure, PTT1 is a pulse-wave propagation time between arbitrary two areas, and α1 and α2 are constants obtained by performing regression analysis.

For example, a blood-pressure estimation model M1-1 is expressed by equation (2) below by using the pulse-wave propagation time PTT (23-24) between area 23 and area 24.

BP1-1=α1-1PTT(23-24)+α2-1 (2)

Also, for example, a blood-pressure estimation model M1-2 is expressed by equation (3) below by using the pulse-wave propagation time PTT (23-33) between area 23 and area 33.

BP1-2=α1-2PTT(23-33)+α2-2 (3)

With respect to all combinations (1326 combinations) of two areas selected from 52 areas extracted by the skin-area extracting unit 16 as the skin areas 161, the blood-pressure estimation model creating unit 30 creates blood-pressure estimation models M1 (M1-1 to M1-1326) with complexity 1.

Next, the blood-pressure estimation model creating unit 30 creates blood-pressure estimation models M2 with complexity 2. That is, the blood-pressure estimation model creating unit 30 creates each blood-pressure estimation model M2 using two pulse-wave propagation times PTT1 and PTT2 as explanatory variables. Specifically, the blood-pressure estimation model creating unit 30 creates a blood-pressure estimation model M2 noted in equation (4) below by performing regression analysis using a least-squares method on two mutually different pulse-wave propagation times PTT1 and PTT2 determined by the pulse-wave parameter determining unit 20 and the subject body's blood pressure obtained by the blood-pressure obtaining unit 2.

BP2=β1PTT1+β2PTT2+β3 (4)

where BP2 is a predicted blood pressure, PTT1 and PTT2 are pulse-wave propagation times between arbitrary two areas that are different from each other, and β1, β2, and β3 are constants obtained by performing regression analysis.

For example, a blood-pressure estimation model M2-1 is expressed by equation (5) below by using the pulse-wave propagation time PTT (23-24) between area 23 and area 24 and the pulse-wave propagation time PTT (23-33) between area 23 and area 33.

BP2-1=β1-1PTT(23-24)+β2-1PTT(23-33)+β3-1 (5)

With respect to all combinations (878475 (=1326C2) combinations) of two pulse-wave propagation times selected from 1326 pulse-wave propagation times determined by the pulse-wave parameter determining unit 20, the blood-pressure estimation model creating unit 30 creates blood-pressure estimation models M2 (M2-1 to M2-878475) with complexity 2.

Thereafter, similarly, the blood-pressure estimation model creating unit 30 creates blood-pressure estimation models M3 with complexity 3, blood-pressure estimation models M4 with complexity 4 . . . . The blood-pressure estimation model creating unit 30 outputs the created blood-pressure estimation models to the blood-pressure estimation model evaluating unit 40 (more specifically, the evaluation predicted-blood-pressure determining unit 41).

Although an aspect in which the blood-pressure estimation model creating unit 30 creates linear blood-pressure estimation models by performing regression analysis has been described in the present embodiment, the blood-pressure measuring device in the present disclosure is not limited thereto. A blood-pressure measuring device in one aspect of the present disclosure may create nonlinear blood-pressure estimation models. For creating the blood-pressure estimation models, estimation considering over-learning suppression not only by regression analysis using the least-squares method but also by Lasso incorporating L1 regularization may be performed.

The blood-pressure estimation model evaluating unit 40 evaluates the blood-pressure estimation models created by the blood-pressure estimation model creating unit 30. The blood-pressure estimation model evaluating unit 40 includes the evaluation predicted-blood-pressure determining unit 41 and the model-evaluation-index determining unit 42.

The evaluation predicted-blood-pressure determining unit 41 determines predicted blood pressures in the blood-pressure estimation models by applying the pulse-wave propagation times PTT, output from the pulse-wave parameter determining unit 20 as data for testing, to the blood-pressure estimation models created by the blood-pressure estimation model creating unit 30.

The model-evaluation-index determining unit 42 determines, as evaluation indices for the blood-pressure estimation models, mean square errors (MSEs: Mean Square errors) between the predicted blood pressures determined by the evaluation predicted-blood-pressure determining unit 41 and the blood pressure (data for testing) obtained by the blood-pressure obtaining unit 2. The model-evaluation-index determining unit 42 determines evaluation indices for the blood-pressure estimation models in order of the blood-pressure estimation models with the lowest complexity first and outputs the evaluation indices to the model selecting unit 50.

The evaluation indices for the blood-pressure estimation models are not limited to the mean square errors, and for example, mean absolute errors, standard deviations of errors, degree-of-freedom adjusted indices of determination, AIC (Akaike's Information Criteria), or the like can be used.

Based on the evaluation performed by the blood-pressure estimation model evaluating unit 40 (more specifically, the model-evaluation-index determining unit 42), the model selecting unit 50 selects a measurement model from among the plurality of blood-pressure estimation models created by the blood-pressure estimation model creating unit 30.

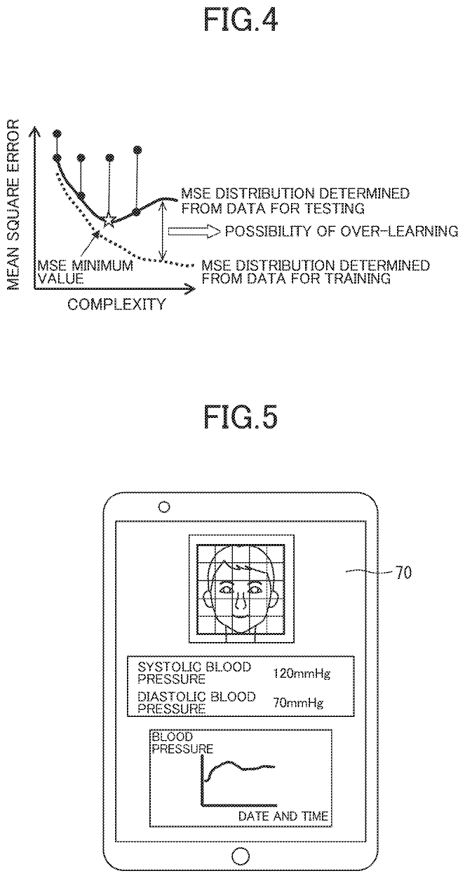

FIG. 4 is a graph for describing a measurement-model selecting method performed by the model selecting unit 50. As shown in FIG. 4, the model selecting unit 50 selects, as a measurement model, a blood-pressure estimation model with which the mean square error has a minimum value when blood-pressure estimation models with which the mean square errors for the respective complexities are the smallest are plotted. The model selecting unit 50 outputs the selected measurement model to the blood-pressure measuring unit 60.

In the blood-pressure measuring device 1A, data for the blood-pressure estimation model creation in the blood-pressure estimation model creating unit 30 (the data for training) and data for the blood-pressure estimation model evaluation in the blood-pressure estimation model evaluating unit 40 (the data for testing) are data that are different from each other. This allows the model selecting unit 50 to select a measurement model that applies well to the data for testing and that has superior generalizability performance, without falling into over-learning, as shown in FIG. 4.

The blood-pressure estimation model creating unit 30 and the blood-pressure estimation model evaluating unit 40 suspend the blood-pressure estimation model creation and the blood-pressure estimation model evaluation, respectively, at a point in time when the model selecting unit 50 selects the measurement model. This makes it possible to reduce the amount of computation in the blood-pressure estimation model creating unit 30 and the blood-pressure estimation model evaluating unit 40.

As described above, the pulse-wave obtaining unit 10, the pulse-wave parameter determining unit 20, the blood-pressure estimation model creating unit 30, the blood-pressure estimation model evaluating unit 40, and the model selecting unit 50 function as the model setting device 100 that sets the measurement model for measuring the blood pressure of the subject body on the basis of pulse waves of the subject body.

The blood-pressure measuring unit 60 measures the blood pressure of the subject body by applying the pulse-wave propagation times PTT, output from the pulse-wave parameter determining unit 20, to the measurement model selected by the model selecting unit 50 (the model setting device 100). The blood-pressure measurement result outputting unit 70 outputs the subject body's blood pressure measured by the blood-pressure measuring unit 60.

FIG. 5 is a view showing one example of the blood-pressure measurement result outputting unit 70. The blood-pressure measurement result outputting unit 70 may be, for example, a display (e.g., a liquid-crystal display), as shown in FIG. 5.

(Processing in Blood-Pressure Measuring Device 1A)

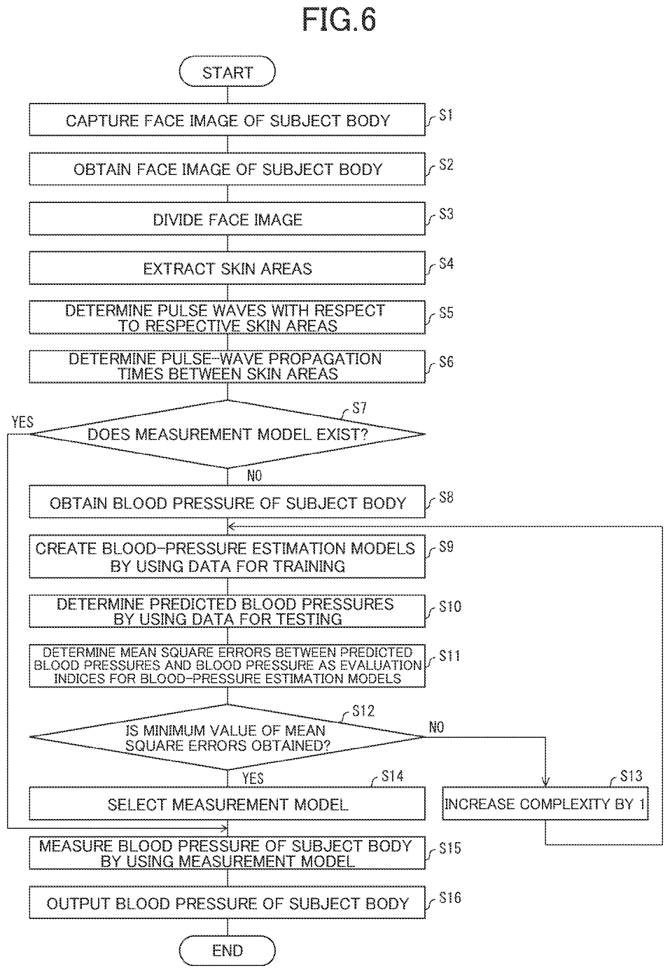

FIG. 6 is a flowchart showing one example of a flow of processing in the blood-pressure measuring device 1A.

As shown in FIG. 6, in subject-body blood-pressure measurement and a model setting method performed by the blood-pressure measuring device 1A, first, the image capture unit 11 captures a face image of a subject body (S1). Next, the face-image obtaining unit 14 obtains a face image of the subject body from the subject body's image captured by the image capture unit 11 (S2). Next, the face-image dividing unit 15 divides the face image, extracted by the face-image obtaining unit 14, into a plurality of areas (S3). Next, the skin-area extracting unit 16 extracts, as the skin areas 161, areas that are included in the areas divided by the face-image dividing unit 15 and in which the skin is not completely hidden (S4). Next, the pulse-wave determining unit 17 determines pulse waves with respect to the respective skin areas 161 extracted by the skin-area extracting unit 16 (S5). Steps S1 to S5 are pulse-wave obtaining processes for obtaining pulse waves in a plurality of areas in the face of the subject body.

Next, by using the pulse waves (pulse wave signals) in the respective skin areas 161, the pulse waves being obtained in step S5, the pulse-wave parameter determining unit 20 determines pulse-wave propagation times PTT between the skin areas 161 (S6, a pulse-wave propagation time determination process, a pulse-wave parameter determining process).

Next, it is checked whether or not a measurement model for the subject body whose blood pressure is currently going to be measured already exists (S7). When the measurement model does not exist (NO in S7), the blood-pressure obtaining unit 2 obtains the blood pressure of the subject body (S8, a blood-pressure obtaining process).

Next, the blood-pressure estimation model creating unit 30 creates a plurality of blood-pressure estimation models with complexity 1 by using the pulse-wave propagation times PTT determined by the pulse-wave parameter determining unit 20 and the subject body's blood pressure obtained by the blood-pressure obtaining unit 2, the pulse-wave propagation times PTT and the subject body's blood pressure being data for training (S9, a blood-pressure estimation model creating process). The subject body's blood pressure used in this process is a blood pressure measured simultaneously with acquiring the face image of the subject body.

Next, the evaluation predicted-blood-pressure determining unit 41 determines predicted blood pressures in the blood-pressure estimation models with complexity 1 by applying the pulse-wave propagation times PTT, output from the pulse-wave parameter determining unit 20 as the data for testing, to the plurality of blood-pressure estimation models with complexity 1 created by the blood-pressure estimation model creating unit 30 (S10).

Next, the model-evaluation-index determining unit 42 determines, as evaluation indices for the blood-pressure estimation models, mean square errors between the predicted blood pressures determined by the evaluation predicted-blood-pressure determining unit 41 and the blood pressure obtained by the blood-pressure obtaining unit 2 (S11). Steps S10 and S11 are blood-pressure estimation model evaluating processes for evaluating the blood-pressure estimation models.

Next, the model selecting unit 50 determines whether or not a minimum value of the mean square errors is obtained when the blood-pressure estimation models with which the mean square errors for the respective complexities are the smallest are plotted (S12). In other words, the model selecting unit 50 determines whether or not the smallest mean square error for the complexity, the smallest mean square error being determined in last step S11, is larger than the smallest mean square error for the complexity, the smallest mean square error being determined in last-but-one step S11. When step S12 is performed for the first time, step S12 indicates NO, since there is no smallest mean square error to be compared.

When the minimum value of the mean square errors is not obtained (in other words, when the smallest mean square error for the complexity, the smallest mean square error being determined in last step S11, is smaller than the smallest mean square error for the complexity, the smallest mean square error being determined in last-but-one step S11) (NO in S12), the complexity for the blood-pressure estimation models is increased by 1 (step S13), and steps S9 to S12 are repeated.

On the other hand, when the minimum value of the mean square errors is obtained (in other words, when the smallest mean square error for the complexity, the smallest mean square error being determined in last step S11, is larger than the smallest mean square error for the complexity, the smallest mean square error being determined in last-but-one step S11) (YES in S12), the model selecting unit 50 selects, as a measurement model, the blood-pressure estimation model with which the mean square error indicates a minimum value (S14). Steps S12 and S14 are model selecting processes for selecting a measurement model from among the plurality of blood-pressure estimation models.

Next, the blood-pressure measuring unit 60 measures the blood pressure of the subject body by applying the pulse-wave propagation times PTT, output from the pulse-wave parameter determining unit 20, to the measurement model selected by the model selecting unit 50 (S15). When a measurement model for the subject body whose blood pressure is currently going to be measured already exists in step S7 (YES in step S7), step S15 is performed without performing steps S8 to S14.

Lastly, the blood-pressure measuring unit 60 outputs a measured blood pressure of the subject body to the blood-pressure measurement result outputting unit 70 (S16).

As described above, the model setting device 100 in the present embodiment creates a plurality of blood-pressure estimation models by using a plurality of pulse-wave propagation times determined from areas that are different from each other. Then, the plurality of blood-pressure estimation models is evaluated, and a measurement model is set.

According to the above-described configuration, a measurement model can be set using pulse-wave propagation times between areas that are highly correlated with the blood pressure of the subject body. As a result, since the model setting device 100 can set a measurement model that suits a vascular network, a contour, the size of the face, and so on that differ from one subject body to another, the blood pressure of the subject body can be measured with high accuracy.

Although a configuration in which the image capture unit 11 is comprised by the blood-pressure measuring device 1A has been described in the present embodiment, the blood-pressure measuring device in the present disclosure is not limited thereto. One aspect of the present disclosure may be an aspect in which an image captured with a built-in camera of a smartphone, a camera included in a monitoring robot, or the like is output to a blood-pressure measuring device, and a measurement model is set using the image.

Also, an aspect in which a face image of a subject body is used to set a measurement model has been described in the present embodiment, the blood-pressure measuring device in the present disclosure is not limited thereto. In one aspect of the present disclosure, a measurement model may be set using an image of an area other than the face, as long as it is an area from which the pulse wave of a subject body can be obtained. However, when a face image is used, load on the subject body is small, and it is possible to measure the blood pressure when the subject body is in a natural state.

Also, although, in the present embodiment, pulse waves are obtained without contacting the living body by using the camera, the present disclosure is not limited thereto. In the blood-pressure measuring device in the present disclosure, it is sufficient that pulse waves can be obtained from at least three areas of the subject body, and pulse waves may be obtained using a contact-type sensor.

Also, although an aspect in which blood-pressure estimation models are respectively created with respect to all combinations of the pulse-wave propagation times PTT determined by the pulse-wave parameter determining unit 20 for each complexity has been described in the present embodiment, the blood-pressure measuring device in the present disclosure is not limited thereto. In one aspect of the present disclosure, at least two blood-pressure estimation models with different complexities may be created using at least two pulse-wave propagation times PTT.

Also, although, in the present embodiment, the data for the blood-pressure estimation model creation in the blood-pressure estimation model creating unit 30 (the data for training) and the data for the blood-pressure estimation model evaluation in the blood-pressure estimation model evaluating unit 40 (the data for testing) are data that are different from each other, the blood-pressure measuring device in the present disclosure is not limited thereto. In one aspect of the present disclosure, the data for training and the data for evaluation can be made to be the same data when the evaluation of the blood-pressure estimation models and selection of the measurement model are performed using indices that can be determined from the data used in the blood-pressure estimation model creating unit 30 (e.g., degree-of-freedom adjusted coefficients of determination or the like).

Also, although an aspect in which the blood-pressure estimation model creating unit 30 creates a plurality of models with different complexities has been described in the present embodiment, the present disclosure is not limited to this aspect. In one aspect of the present disclosure, model creation may be performed as described below. That is, predicted blood pressures are determined by applying the pulse wave parameters, output by the pulse-wave parameter determining unit 20, as the data for training to one model created using the data for training. Next, the data for training is classified according to the polarities and the sizes of errors in the determined predicted blood pressures for the data for training relative to the blood pressure obtained by the blood-pressure obtaining unit 2, and model creation is performed for each classification by using data corresponding to the classification. Specifically, for example, when error 0 is set for a threshold, the data for training is classified into two groups, that is, a group (1) of positive errors and a group (2) of negative errors, and model creation is performed for each classification. As a result, even for data with which the degree of conformance is low and error increases with respect to one model, data having a similar error tendency (e.g., a group of data with which a positive error occurs with respect to one model) is newly re-learned as the same classification to thereby make it possible to create a model that can handle various data tendencies. Models created with the group (1) of positive errors and models created with the group (2) of negative errors may be blood-pressure estimation models using different parameters.

Also, although an aspect in which the model selecting unit 50 performs model selection based on the model evaluation indices determined from the data for testing which includes a plurality of pieces of subject-person data determined by the blood-pressure estimation model evaluating unit 40, and model selection with high generalizability for subject people is performed has been described in the present embodiment, the present disclosure is not limited thereto. In one aspect of the present disclosure, an optimum model may be selected for each subject person by using at least one piece of data of each subject person.

Also, although, in the present embodiment, the plurality of pulse-wave propagation times PTT determined from areas that are different from each other is used as explanatory variables (pulse wave parameters) in order to create the blood-pressure estimation models, the blood-pressure measuring device in the present disclosure is not limited thereto. In one aspect of the present disclosure, in addition to the pulse-wave propagation times PTT, waveform features of pulse waves determined from the respective skin areas 161 may be as explanatory variables for the blood-pressure estimation models to create the blood-pressure estimation models. Also, in one aspect of the present disclosure, without using the pulse-wave propagation times PTT, only a plurality of waveform features may be used as explanatory variables for the blood-pressure estimation models to create the blood-pressure estimation models. Also, in one aspect of the present disclosure, for example, the numbers of pulses, other than the pulse-wave propagation times and the waveform features, can be used as the pulse wave parameters.

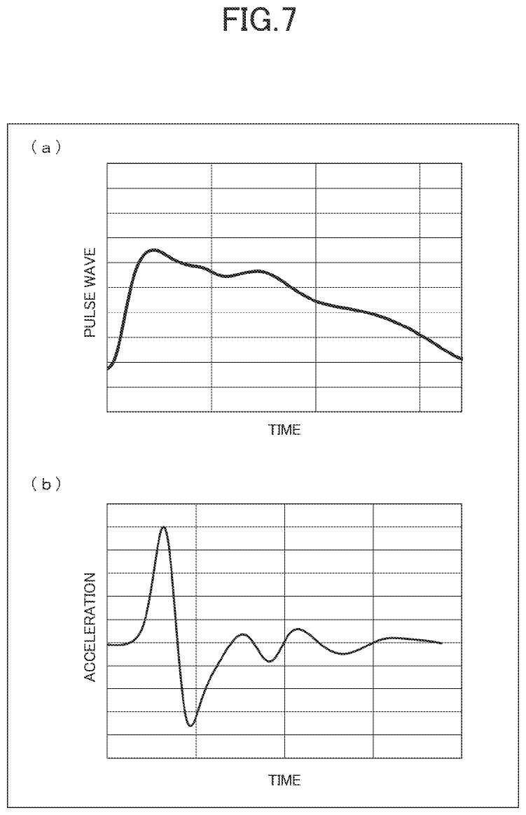

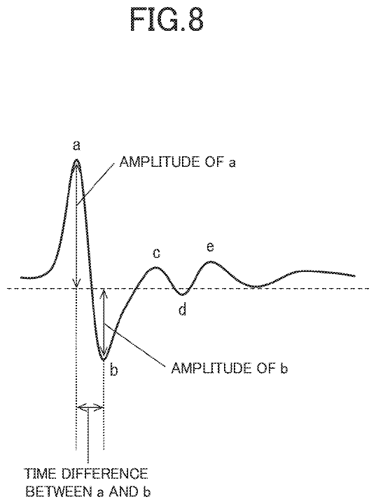

(a) in FIG. 7 is a graph showing one example of a pulse wave waveform, and (b) in FIG. 7 is a graph showing one example of an acceleration pulse wave waveform. FIG. 8 is a graph for describing the aforementioned waveform features. The aforementioned waveform features can be determined using a pulse wave waveform as shown in (a) in FIG. 7 or an acceleration pulse wave waveform obtained by performing differentiation on a pulse wave signal twice, as shown in (b) in FIG. 7. For example, as shown in FIG. 8, amplitudes at feature points a to e, the ratio of the amplitudes (e.g., a ratio of the amplitude of feature point a to the amplitude of feature point b), a time difference between the waveform features (e.g., a time difference between feature point a and feature point b), or the like can be used as the waveform features.

For a model that uses only the pulse-wave propagation times PTT described above in the present embodiment, the pulse waves need to be determined in at least three areas in order to determine the plurality of pulse-wave propagation times. In contrast, when only waveform features are used, a plurality of waveform features can be determined from one area, and thus it is sufficient that the pulse wave be determined in at least one area. Also, when the pulse-wave propagation times PTT and the waveform features are used, determining the pulse waves in at least two areas makes it possible to obtain one pulse-wave propagation time PTT and a plurality of waveform features.

### Second Embodiment

Another embodiment of the present invention will be described below. For convenience of description, members having the same functions as those of the members described in the above-described embodiment are denoted by the same numerals, and descriptions thereof are not repeated.

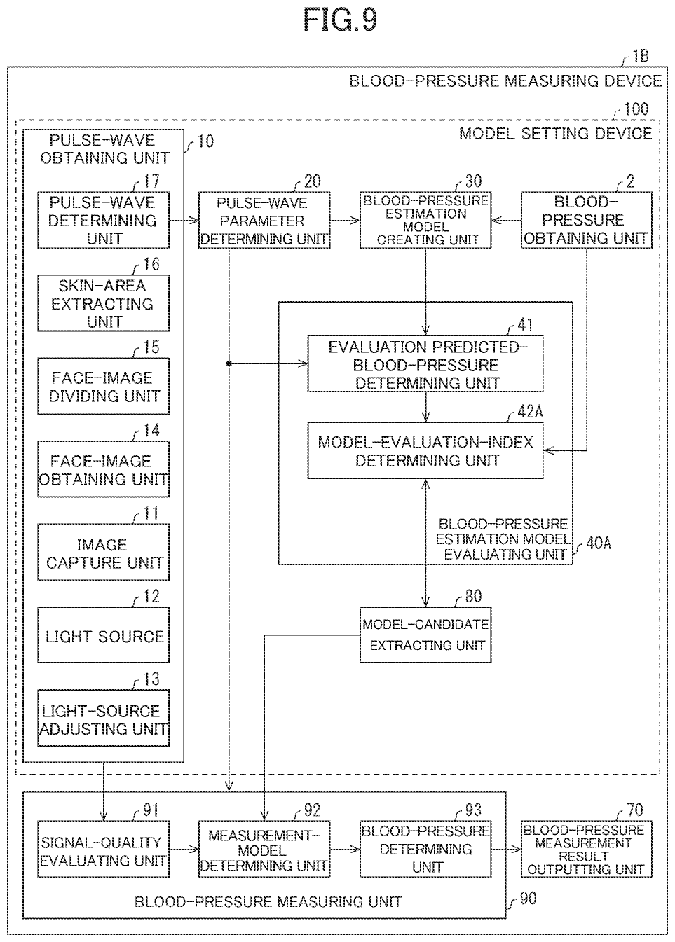

FIG. 9 is a block diagram showing the configuration of a blood-pressure measuring device 1B in the present embodiment. As shown in FIG. 9, the blood-pressure measuring device 1B comprises a blood-pressure estimation model evaluating unit 40A, a model-candidate extracting unit 80, and a blood-pressure measuring unit 90 in addition to the blood-pressure estimation model evaluating unit 40, the model selecting unit 50, and the blood-pressure measuring unit 60 in the first embodiment.

The blood-pressure estimation model evaluating unit 40A comprises a model-evaluation-index determining unit 42A instead of the model-evaluation-index determining unit 42 in the first embodiment.

The model-evaluation-index determining unit 42A determines, as evaluation indices for the blood-pressure estimation models, standard deviations of errors between predicted blood pressures determined by the evaluation predicted-blood-pressure determining unit 41 and the blood pressure (data for testing) obtained by the blood-pressure obtaining unit 2. The model-evaluation-index determining unit 42A outputs the determined evaluation indices to the model-candidate extracting unit 80.

The model-candidate extracting unit 80 extracts blood-pressure estimation models with which the evaluation indices determined by the model-evaluation-index determining unit 42 have values that are smaller than a certain threshold as measurement model candidates for blood-pressure measurement in the blood-pressure measuring unit 90. The model-candidate extracting unit 80 has a function of a model selecting unit that selects a plurality of measurement model candidates for the blood-pressure measurement in the blood-pressure measuring unit 90.

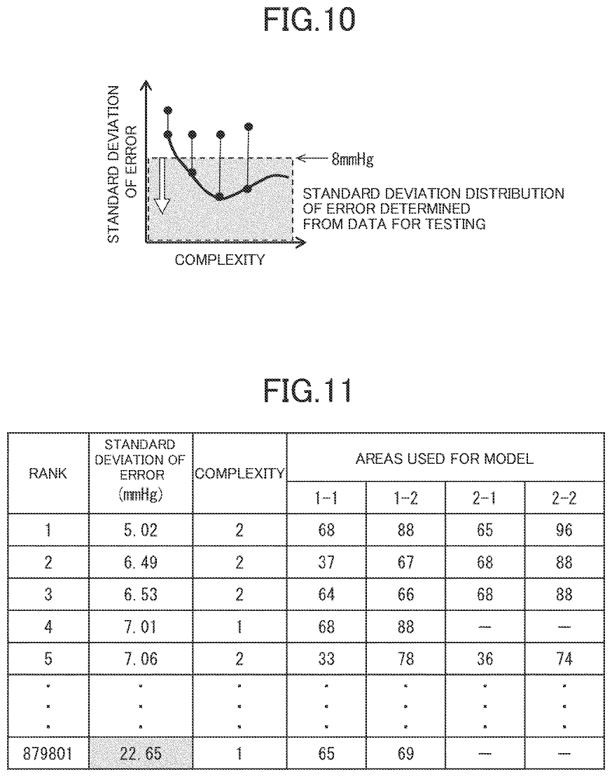

FIG. 10 is a graph showing a distribution of standard deviations of errors in the blood-pressure estimation models, the standard deviations being determined by the model-evaluation-index determining unit 42A by using the data for testing.

As shown in FIG. 10, the model-candidate extracting unit 80 extracts, as measurement model candidates, for example, blood-pressure estimation models with which the standard deviations of errors are smaller than or equal to 8 mmgHg, which is a standard for non-invasive sphygmomanometers.

FIG. 11 is a table showing rankings of standard deviations of errors determined by the model-evaluation-index determining unit 42A. An example in which blood-pressure estimation models with complexity 1 or 2 will be described in the present embodiment. As shown in FIG. 11, the model-evaluation-index determining unit 42A obtains standard errors of errors in 879801 blood-pressure estimation models, which are a total of 1326 blood-pressure estimation models with complexity 1 and 878475 blood-pressure estimation models with complexity 2. For example, the blood-pressure estimation model at rank 1 is a blood-pressure estimation model with complexity 2 which uses a pulse-wave propagation time PTT (68-88) between area 68 and area 88 and a pulse-wave propagation time PTT (65-96) between area 65 and area 96, and the standard deviation of error is 5.02 mmHg. The model-candidate extracting unit 80 extracts a plurality of blood-pressure estimation models with which the standard deviations of errors are 8 mmgHg or less from among 879801 blood-pressure estimation models as measurement model candidates and outputs the extracted measurement model candidates to the blood-pressure measuring unit 90 (more specifically, a measurement-model determining unit 92).

The blood-pressure measuring unit 90 comprises a signal-quality evaluating unit 91, the measurement-model determining unit 92, and a blood-pressure determining unit 93.

The signal-quality evaluating unit 91 evaluates the signal qualities of pulse waves in the respective areas used during measurement of the blood pressure. Specifically, the signal-quality evaluating unit 91 determines SNRs (signal-to-noise ratios, Signal-to-Noise Ratios) of pulse wave signals determined with the method described below.

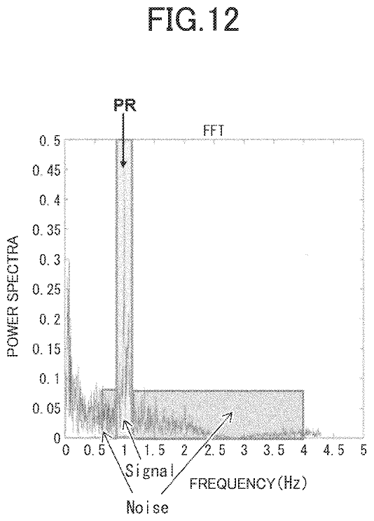

FIG. 12 is a graph showing one example of power spectra of pulse wave signals.

The premise is that the pulse wave signals have a certain cycle that matches the heart rate since the pulse wave is a wave that is transmitted to arteries owing to pumping actions of the heart, and a peak (PR) can be confirmed around 1 Hz in rest-time data when frequency analysis is performed on the pulse wave signals, as shown in FIG. 12. By utilizing this, the signal-quality evaluating unit 91 determines SNR=Signal/Noise in the power spectra in the frequencies of pulse wave signals, where the sum of power at ±0.05 Hz of PR is represented by Signal t, and the sum of power at 0.75 to 4.0 Hz other than the Signal band is represented by Noise, as shown in FIG. 12. The signal-quality evaluating unit 91 outputs the determined SNRs to the measurement-model determining unit 92. The bandwidth of Signal and the bandwidth of Noise are not limited to the above-described widths and can be determined as appropriate.

Based on the pulse-wave signal qualities obtained by the signal-quality evaluating unit 91, the measurement-model determining unit 92 determines a measurement model from among the plurality of measurement model candidates extracted by the model-candidate extracting unit 80. Specifically, the measurement-model determining unit 92 determines, as a measurement model, a measurement model candidate with which the SNR in each area used for the measurement model candidate is larger than or equal to 0.15 in all areas, the measurement model candidate being included in the measurement model candidates extracted by the model-candidate extracting unit 80.

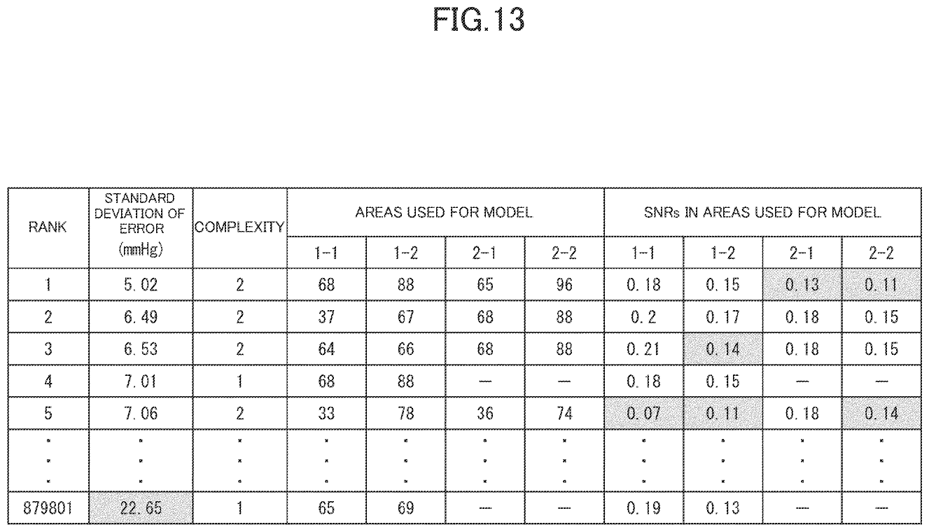

FIG. 13 is a table for describing a measurement-model determining method performed by the measurement-model determining unit 92. In the example shown in FIG. 13, in a measurement model candidate at rank 2 and a measurement model candidate at rank 4, (condition 1) the standard deviations of errors are smaller than or equal to 8 mmgHg, and (condition 2) the SNR in each area is larger than or equal to 0.15 in all areas. In this case, the measurement-model determining unit 92 determines the measurement model candidate at rank 2, which is a higher rank, as a measurement. The measurement-model determining unit 92 outputs the determined measurement model to the blood-pressure determining unit 93. Although the threshold for the SNRs is 0.15 in the present embodiment, the threshold for the SNRs is not limited thereto and may be set as appropriate.

The blood-pressure determining unit 93 measures the blood pressure of the subject body by applying the pulse-wave propagation times PTT, output from the pulse-wave parameter determining unit 20, to the measurement-model determined by the measurement-model determining unit 92. The subject body's blood pressure measured by the blood-pressure determining unit 93 (the blood-pressure measuring unit 90) is output by the blood-pressure measurement result outputting unit 70.

As described above, in the blood-pressure measuring device 1B in the present embodiment, the blood-pressure measuring unit 90 selects a measurement model from among the plurality of measurement models candidates extracted by the model-candidate extracting unit 80, based on the evaluation performed by the blood-pressure estimation model evaluating unit 40 (more specifically, the model-evaluation-index determining unit 42A) and the pulse-wave signal qualities obtained by the signal-quality evaluating unit 91, and measures the blood pressure of the subject body.

According to the above-described configuration, when the blood-pressure measuring unit 90 measures the blood pressure of the subject body, a measurement model candidate with which the signal quality of the pulse waves is high at the time of the measurement can be used from among the plurality of measurement model candidates as a measurement model. As a result, even when an image capture environment at the time of creating the measurement model and an image capture environment at the time of measuring the blood pressure differ from each other significantly, the blood pressure can be measured using an appropriate measurement model corresponding to the image capture environment. This makes it possible to reliably perform high-accuracy blood-pressure measurement.

An aspect in which a measurement model candidate having a higher rank is determined as a measurement when there is a plurality of measurement model candidates that satisfy both conditions 1 and 2 has been described in the present embodiment, the blood-pressure measuring device in the present disclosure is not limited thereto. A blood-pressure measuring device in one aspect of the present disclosure may have an aspect in which it determines a plurality of blood pressures by using measurement model candidates that satisfy both conditions 1 and 2 and determines a representative value (e.g., an average value or a median) of the plurality of blood pressures as the blood pressure.

Also, although an aspect in which the rankings of the standard deviations of errors determined by the model-evaluation-index determining unit 42A are created, and the measurement model is determined from the rankings has been described in the present embodiment, the blood-pressure measuring device in the present disclosure is not limited thereto. The blood-pressure measuring device in one aspect of the present disclosure may have an aspect in which rankings of the respective areas are created using the signal qualities evaluated by the signal-quality evaluating unit 91, and a measurement model candidate using a higher-ranking area is determined from among the measurement model candidates as the measurement model.

Also, although an aspect in which the blood-pressure estimation models with complexity 1 or 2 are used has been described above in the present embodiment, the blood-pressure measuring device in the present disclosure is not limited thereto. The blood-pressure measuring device in one aspect of the present disclosure may have an aspect in which, for example, when it is desirable to measure a blood pressure in real time, for example, only blood-pressure estimation models with low complexity (e.g., only blood-pressure estimation models with complexity 1) are used in order to reduce the amount of computation.

Also, although an aspect in which the signal-quality evaluating unit 91 evaluates the signal qualities of pulse waves by using SNRs of the pulse wave signals has been described in the present embodiment, the blood-pressure measuring device in the present disclosure is not limited thereto. In one aspect of the present disclosure, the signal-quality evaluating unit 91 may evaluate the signal qualities of the pulse waves by using luminance values.

### Implementation Example Using Software

The control blocks in the blood-pressure measuring device 1A and the blood-pressure measuring device 1B (particularly, the pulse-wave obtaining unit 10, the pulse-wave parameter determining unit 20, the blood-pressure estimation model creating unit 30, the blood-pressure estimation model evaluating unit 40, the model selecting unit 50, and the blood-pressure measuring unit 60) may be realized by logic circuits (hardware) formed in integrated circuits (IC chips) or the like or may be realized by software.

In the latter case, the blood-pressure measuring device 1A and the blood-pressure measuring device 1B each comprise a computer for executing instructions from a program that is software for realizing the individual functions. This computer comprises, for example, at least one processor (control device) and also comprises at least one computer-readable recording medium that stores the above-described program therein. In the above-described computer, the processor reads the program from the recording medium and executes the program to thereby achieve the object of the present invention. For example, a CPU (central processing unit) can be used as the above-described processor. A “non-transient tangible medium”, for example, a tape, a disk, a card, a semiconductor memory, a programmable logic circuit, or the like, in addition to a ROM (read-only memory) or the like can be used as the recording medium. Also, the computer may further comprise a RAM (random access memory) or the like to which the above-described program is loaded. Also, the program may be supplied to the computer over an arbitrary transmission medium (a communications network, a broadcast radio wave, or the like) through which the program can be transmitted. One aspect of the present invention can be realized in the form of data signals embodied by electronic transmission of the above-described program and embedded in carrier waves.

The present invention is not limited to each embodiment described above, various changes are possible within the scope recited in the appended claims, and embodiments obtained by appropriately combining the technical means respectively disclosed in the different embodiments are also encompassed by the technical scope of the present invention. In addition, new technical features can be formed by combining the technical means respectively disclosed in the embodiments.

### CROSS REFERENCE TO RELATED APPLICATION

This application claims the benefit of priority to Japanese Patent Application No. 2018-091651 filed on May 10, 2018, the entire contents of which are herein incorporated by reference.

### REFERENCE SIGNS LIST

1A, 1B blood-pressure measuring device
2 blood-pressure obtaining unit
10 pulse-wave obtaining unit
12 light source
13 light-source adjusting unit
20 pulse-wave parameter determining unit (pulse-wave propagation time determining unit)
30 blood-pressure estimation model creating unit
40, 40A blood-pressure estimation model evaluating unit
50 model selecting unit
60, 90 blood-pressure measuring unit
80 model-candidate extracting unit (model selecting unit)
91 signal-quality evaluating unit
100 model setting device

1. A model setting device that sets a measurement model for measuring a blood pressure of a living body based on pulse waves of the living body, the model setting device comprising:
a blood-pressure obtaining unit that obtains a blood pressure of the living body;
a pulse-wave obtaining unit that obtains the pulse waves in an area on a body surface of the living body;
a pulse-wave parameter determining unit that determines a plurality of pulse wave parameters by using the pulse waves obtained by the pulse-wave obtaining unit;
a blood-pressure estimation model creating unit that creates a plurality of blood-pressure estimation models for estimating a blood pressure of the living body by using the plurality of pulse wave parameters determined by the pulse-wave parameter determining unit and the living body's blood pressure obtained by the blood-pressure obtaining unit;
a blood-pressure estimation model evaluating unit that evaluates the plurality of blood-pressure estimation models created by the blood-pressure estimation model creating unit; and
a model selecting unit that selects at least one measurement model from among the plurality of blood-pressure estimation models, based on the evaluation performed by the blood-pressure estimation model evaluating unit.

2. The model setting device according to claim 1,
wherein the pulse-wave obtaining unit obtains the pulse waves in two or more areas on the body surface of the living body; and
the pulse-wave parameter determining unit determines at least one pulse-wave propagation time between areas of the two or more areas and at least one waveform feature as the pulse wave parameters by using the pulse waves obtained by the pulse-wave obtaining unit.

3. The model setting device according to claim 1,
wherein the pulse-wave obtaining unit obtains the pulse waves in three or more areas on the body surface of the living body; and
the pulse-wave parameter determining unit determines pulse-wave propagation times between two areas of the three or more area as the pulse wave parameters by using the pulse waves obtained by the pulse-wave obtaining unit.

4. The model setting device according to claim 2,
wherein the pulse-wave parameter determining unit determines the pulse-wave propagation times with respect to all combinations of two areas selected from areas extracted as skin areas of the living body.

5. The model setting device according to claim 1,
wherein the pulse-wave obtaining unit obtains the pulse waves in one or more areas on the body surface of the living body; and
the pulse-wave parameter determining unit determines waveform features in the one or more areas as the pulse wave parameters by using the pulse waves obtained by the pulse-wave obtaining unit.

6. The model setting device according to claim 1, wherein the pulse-wave obtaining unit obtains the pulse waves in a face of the living body.

7. A blood-pressure measuring device comprising:
a model setting device according to claim 1; and
a blood-pressure measuring unit that measures the blood pressure of the living body by using the measurement model selected by the model selecting unit.

8. The blood-pressure measuring device according to claim 7, further comprising:
a signal-quality evaluating unit that evaluates a signal quality of the pulse waves obtained by the pulse-wave obtaining unit,
wherein the model selecting unit selects a plurality of candidates for the measurement model; and
the blood-pressure measuring unit selects the measurement model from among the plurality of candidates selected by the model selecting unit, based on the evaluation performed by the blood-pressure estimation model evaluating unit and the signal quality evaluation performed by the signal-quality evaluating unit, and measures the blood pressure of the living body.

9. The blood-pressure measuring device according to claim 7, comprising:
a light source that illuminates the living body with light when the pulse-wave obtaining unit obtains the living body's pulse waves; and
a light-source adjusting unit that adjusts the light source in order to accurately determine the pulse wave parameters used for the measurement model selected by the model selecting unit.

10. A model setting method that sets a measurement model for measuring a blood pressure of a living body based on pulse waves of the living body, the model setting method including:
a blood-pressure obtaining process of obtaining a blood pressure of the living body;
a pulse-wave obtaining process of obtaining the pulse waves in an area on a body surface of the living body;
a pulse-wave parameter determining process of determining a plurality of pulse wave parameters by using the pulse waves obtained in the pulse-wave obtaining process;
a blood-pressure estimation model creating process of creating a plurality of blood-pressure estimation models for estimating a blood pressure of the living body by using the plurality of pulse wave parameters determined in the pulse-wave parameter determining process and the living body's blood pressure obtained in the blood-pressure obtaining process;
a blood-pressure estimation model evaluating process of evaluating the plurality of blood-pressure estimation models created in the blood-pressure estimation model creating process; and
a model selecting process of selecting at least one measurement model from among the plurality of blood-pressure estimation models, based on the evaluation performed in the blood-pressure estimation model evaluating process.

11. The model setting device according to claim 3,
wherein the pulse-wave parameter determining unit determines the pulse-wave propagation times with respect to all combinations of two areas selected from areas extracted as skin areas of the living body.

12. The model setting device according to claim 2,
wherein the pulse-wave obtaining unit obtains the pulse waves in a face of the living body.

13. The model setting device according to claim 3,
wherein the pulse-wave obtaining unit obtains the pulse waves in a face of the living body.

14. The model setting device according to claim 4,
wherein the pulse-wave obtaining unit obtains the pulse waves in a face of the living body.

15. The model setting device according to claim 5,
wherein the pulse-wave obtaining unit obtains the pulse waves in a face of the living body.

16. The model setting device according to claim 11,
wherein the pulse-wave obtaining unit obtains the pulse waves in a face of the living body.

17. The blood-pressure measurement device according to claim 8, comprising:
a light source that illuminates the living body with light when the pulse-wave obtaining unit obtains the pulse waves of the living body; and
a light-source adjusting unit that adjusts the light source in order to accurately determine the pulse wave parameters used for the measurement model selected by the model selecting unit.
